(19) United States
(12) Patent Application Publication
(10) Pub. No.: US 2022/0125303 A1
(43) Pub. Date: Apr. 28, 2022

(54) SYSTEMS AND METHODS FOR AXIAL LENGTH DERIVATION

(71) Applicant: VISIONIX - LUNEAU TECHNOLOGY, Jerusalem (IL)

(72) Inventors: Marc ABITBOL, Jerusalem (IL); Ian MELNICK, Jerusalem (IL); Haniel ABITBOL, Jerusalem (IL)

(73) Assignee: VISIONIX - LUNEAU TECHNOLOGY, Jerusalem (IL)

(21) Appl. No.: 17/291,384

(22) PCT Filed: Nov. 7, 2018
(86) PCT No.: PCT/IL18/51190

§ 371 Date: May 5, 2021

(51) Int. Cl. A61B 3/103; A61B 3/10; A61B 3/107

(52) U.S. Cl. CPC A61B 3/103; A61B 3/107; A61B 3/1015

(57) ABSTRACT

An ocular measurement system including an integrated measurement subsystem including at least a refraction measurement module for measuring at least refraction of an eye of a subject and a corneal measurement module integrated with the refraction measurement module for measuring at least a thickness and a shape of a cornea of the eye and an axial length finding subsystem for finding an axial length of the eye, based on taking into account at least the refraction of the eye and the thickness and the shape of the cornea.

### FIELD OF THE INVENTION

The present invention relates generally to ocular measurement devices and more particularly to ocular measurement devices for indirect derivation of the axial length of the eye.

### BACKGROUND OF THE INVENTION

Various types of devices for indirect derivation of the axial length of the eye are known in the art.

### SUMMARY OF THE INVENTION

The present invention seeks to provide reliable and cost-effective systems and methods for finding the axial length of the eye, without requiring direct measurement thereof. There is thus provided in accordance with a preferred embodiment of the present invention an ocular measurement system including an integrated measurement subsystem including at least a refraction measurement module for measuring at least refraction of an eye of a subject and a corneal measurement module integrated with the refraction measurement module for measuring at least a thickness and a shape of a cornea of the eye and an axial length finding subsystem for finding an axial length of the eye, based on taking into account at least the refraction of the eye and the thickness and the shape of the cornea.

In accordance with a preferred embodiment of the present invention, the finding the axial length of the eye additionally includes taking into account a modelled value of at least one parameter of a lens of the eye.

Preferably, the at least one parameter of the lens includes at least one of a thickness of the lens, a radius of an anterior surface of the lens, a radius of a posterior surface of the lens and a refractive index of the lens.

Preferably, the refraction measurement module includes a Shack-Hartmann system for analyzing a wavefront of light reflected from the eye.

Preferably, the corneal measurement module includes at least a pachymeter and a corneal topographer.

Preferably, the pachymeter measures at least one of a thickness of the cornea, a posterior radius of the cornea and an anterior chamber depth of the eye.

Preferably, the corneal topographer measures at least an anterior radius of the cornea.

There is also provided in accordance with another preferred embodiment of the present invention an ocular measurement system including a refraction measurement subsystem for measuring refraction of an eye of a subject, a corneal measurement subsystem for measuring at least a thickness and a shape of a cornea of the eye and an axial length calculation subsystem for performing a formulaic calculation for calculating an axial length of the eye, based on taking into account at least the refraction of the eye, the thickness and the shape of the cornea and a modelled value of at least one parameter of a lens of the eye.

Preferably, the at least one parameter of the lens includes at least one of a thickness of the lens, a radius of an anterior surface of the lens, a radius of a posterior surface of the lens and a refractive index of the lens.

Preferably, the formulaic calculation includes at least one formulaic calculation for finding at least one of a power of the cornea, a power of the lens, a combined power of the cornea and the lens and a distance between a principal point of the eye and a focal point of the eye.

Preferably, the power of the cornea is calculated in accordance with P_{c}=P_{1c}+P_{2c}−[CT×(P_{1c}×P_{2c})/n_{cornea}], where P_{c} is the power of the cornea, P_{1c}=(N_{cornea}−nₐᵢᵣ)/CRₐ, P_{2c}=(n_{aqueous}−N_{cornea})/CRₚ, CT is a thickness of the cornea, n_{cornea} is a refractive index of the cornea, nₐᵢᵣ is a refractive index of air, CRₐ is an anterior radius of the cornea, n_{aqueous} is a refractive index of an aqueous humour of the eye and CR_{P} is a posterior radius of the cornea.

Preferably, the power of the lens is calculated in accordance with P_{L}=P_{1L}+P−_{2L}−(LT×(P_{1L}×P_{2L})/nₗₑₙₛ] where P_{L} is the power of the lens, P_{1L}=(nₗₑₙₛ−n_{aqueous})/LRₐ, P_{2L}=(Nᵥᵢₜᵣₑₒᵤₛ−nₗₑₙₛ)/LR_{P}, LT is a thickness of the lens, LRₐ is an anterior radius of the lens, LR_{P} is a posterior radius of lens, nₗₑₙₛ is a refractive index of the lens and nᵥᵢₜᵣₑₒᵤₛ is a refractive index of a vitreous humour of the eye.

Preferably, the combined power of the cornea and the lens is calculated in accordance with Pₜₒₜₐₗ=P_{c}+P_{L}−[d×(P_{c}×P_{L})/n_{aqueous}], where Pₜₒₜₐₗ is the combined power of the cornea and the lens, d is an effective length of a cornea lens system and is calculated in accordance with d=dₜₒₜₐₗ−PPₗₑₙₛ−PP_{cornea}, wherein dₜₒₜₐₗ=CT+ACD+LT, wherein ACD is an anterior chamber depth, PPₗₑₙₛ is a principal point of the lens, defined as PPₗₑₙₛ=(nᵥᵢₜᵣₑₒᵤₛ/P_{L})×(LT/nₗₑₙₛ)×P_{1L}, and PP_{cornea} is a principal point of the cornea, defined as PP_{cornea}=(nₐᵢᵣ/P_{c})×(CT/n_{cornea})×P_{2c}.

Preferably, the distance between a principal point of the eye and a focal point of the eye is calculated in accordance with V=nᵥᵢₜᵣₑₒᵤₛ/(Pₜₒₜₐₗ−(nₐᵢᵣ/u), where V is the distance between a principal point of the eye and a focal point of the eye and u is a reciprocal of a sphere average of the eye.

There is furthermore provided in accordance with yet another preferred embodiment of the present invention an ocular measurement system including a refraction measurement subsystem for measuring refraction of an eye of a subject and providing a measured refraction output, a corneal measurement subsystem for measuring at least a thickness and a shape of a cornea of the eye and providing a measured corneal thickness and shape output and an axial length finding subsystem receiving at least the measured refraction output and the measured corneal thickness and shape output and employing machine learning for finding an axial length of the eye.

In accordance with a preferred embodiment of the present invention, the refraction measurement subsystem is integrated with the corneal measurement subsystem.

Preferably, the machine learning includes employing an artificial neural network.

Additionally or alternatively, the machine learning includes deep learning.

Preferably, the refraction measurement subsystem includes a Shack-Hartmann system for analyzing a wavefront of light reflected from the eye and the corneal measurement subsystem includes at least a pachymeter and a corneal topographer.

Preferably, the axial length finding subsystem is additionally provided with other input parameters relating to the subject, the other input parameters including at least one of an age of the subject, a gender of the subject and an anterior chamber depth of the eye of the subject.

### BRIEF DESCRIPTION OF THE DRAWINGS

The present invention will be understood and appreciated more fully based on the following detailed description taken in conjunction with the drawings in which:

FIG. 1 is a simplified schematic partially pictorial, partially block diagram illustration of an ocular measurement device useful for deriving the axial length of the eye of a subject, constructed and operative in accordance with a preferred embodiment of the present invention;

FIG. 2 is a simplified schematic illustration of the eye of a subject indicating parameters respectively measured, estimated and derived by a system of the type shown in FIG. 1, constructed and operative in accordance with a preferred embodiment of the present invention;

FIG. 3 is a simplified schematic illustration of the eye of a subject, as modelled based on taking into account parameters indicated in FIG. 2;

FIGS. 4A and 4B are simplified flow charts respectively illustrating steps in the derivation of the axial length of the eye of a subject, based on taking into account parameters indicated in FIG. 2; and

FIG. 5 is a simplified schematic illustration of a neural network forming part of a system of the type shown in FIG. 1, constructed and operative in accordance with another preferred embodiment of the present invention.

### DETAILED DESCRIPTION OF PREFERRED EMBODIMENTS

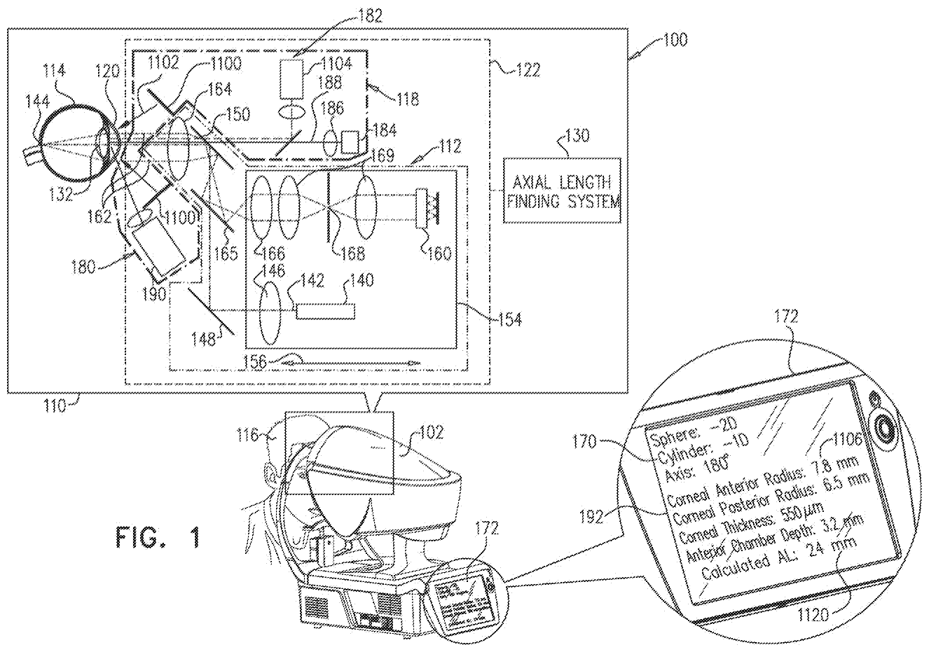

Reference is now made to FIG. 1, which is a simplified schematic partially pictorial, partially block-diagram illustration of an ocular measurement device useful for deriving the axial length of the eye of a subject, constructed and operative in accordance with a preferred embodiment of the present invention.

As seen in FIG. 1, there is provided an ocular measurement system 100, preferably included in an ocular measurement device 102. Here, by way of example only, ocular measurement device 102 is seen to be embodied as a device resembling the VX120 device, commercially available from Luneau, of Chartres, France, although it is appreciated that ocular measurement device 102 may be embodied as any ocular measurement device capable of performing the various measurements and derivations described henceforth.

As best seen at an enlargement 110, ocular measurement system 100 preferably includes a refraction measurement module 112 for measuring the refraction of an eye 114 of a subject 116 and corneal measurement module 118 for measuring at least a thickness and a shape of a cornea 120 of eye 114. Refraction measurement module 112 and corneal measurement module 118 are preferably integrated with one another to form a compact, integrated measurement subsystem 122. Particularly preferably, integrated measurement subsystem 122 may be an integrated measurement system of the type described in U.S. Pat. Nos. 9,220,407 and 9,192,296, assigned to the same assignee as the present invention, the disclosures of which are hereby incorporated by reference in their entirety.

Parameters measured by refraction measurement module 112 and corneal measurement module 118, including at least the refraction of eye 114 and the thickness and shape of cornea 120, are preferably provided to an axial length finding subsystem 130. Axial length finding subsystem 130 preferably, although not necessarily, also forms a part of ocular measurement device 102. The measured parameters output by integrated measurement system 122 are preferably taken into account by axial length finding subsystem 130 for deriving the axial length of eye 114 without requiring direct measurement of the axial length, in accordance with various methods further detailed henceforth.

It is appreciated that refraction measurement module 112 and corneal measurement module 118 integrated therewith may be pre-existing measurement systems present in an ocular measurement device performing ocular testing and functionalities other than axial measurement calculation. By way of example, such an ocular measurement device may be of the type described in U.S. Pat. Nos. 9,220,407 and 9,192,296, the disclosures of which are hereby incorporated by reference in their entirety. The addition of axial length finding subsystem 130 to a preexisting measurement system, such as measurement subsystem 122, thus facilitates the derivation of axial length based on measurements generated by a non-dedicated system that may be primarily operative to perform other functionalities, thereby allowing the axial length to be reliably, conveniently and cost effectively found. This is in contrast to conventional axial length measurement instrumentation, which is typically specialized and highly costly.

Here, by way of example, refraction measurement module 112 is shown to include a laser assembly 140 outputting a laser beam 142, which laser beam 142 is preferably focused on a retina 144 of eye 114 by way of a focusing path preferably comprising a first lens 146, a mirror 148 and a beam splitter 150. Axial movement of a translatable platform 154 supporting laser assembly 140, as indicated by a double-headed arrow 156, serves to compensate for possible variations in the optical power of the eye of a subject, thus ensuring that laser beam 142 remains focused on the retina 144 of the eye 114 of a subject, irrespective of the optical power of eye 114.

The focused laser spot of laser beam 142 on retina 144 forms a point source of light for a Shack-Hartmann sensor 160. Light emanating from the focused laser spot of laser beam 142 on retina 144 preferably propagates back through eye 114, as indicated by a pair of rays 162, collecting various aberration of eye 114 that may be present along the path thereof. Rays 162 preferably propagate, via a second lens 164, towards beam splitter 150, at which beam splitter 150 rays 162 are preferably reflected towards an additional beam splitter 165. Additional beam splitter 165 preferably in turn reflects rays 162 towards a third lens 166, which third lens 166 in combination with second lens 164 forms a first pair of relay lenses. A pin-hole 168 is preferably at the focus of a second pair of relay lenses 169, through which pin-hole 168 and second pair of relay lenses 169 rays 162 are focused towards Shack-Hartmann sensor 160. At Shack-Hartmann sensor 160, features of the wavefront of rays 162 may be used to derive corresponding aberrations of eye 114 and the refraction of the eye 114 calculated based thereon, in accordance with wavefront analysis methods well known in the art. Movement of platform 154, which platform 154 preferably additionally supports Shack-Hartmann sensor 160, preferably serves to ensure that rays 162 remain focused on sensor 160 notwithstanding possible variations in the optical properties of eye 114 of a range of subjects. Refraction measurement subsystem 112 may also include a fixation target for eye fixation during measurement, not shown in FIG. 1 for reasons of clarity.

Here, by way of example, the refractive error of eye 114 of subject 116 as measured by refraction measurement module 112 is shown to be output as ‘Sphere: −2D, Cylinder: −1D and Axis: 180° ’, displayed at a location 170 on a display screen 172 of device 102.

Further by way of example, corneal measurement module 118 is shown to comprise a pachymeter 180 and corneal topographer 182. Pachymeter 180 preferably includes a blue light slit source 184 coupled to a pachymeter lens 186 for focusing a narrow slit of blue light, indicated by a ray 188, on cornea 120 via second lens 164. The incident light 188 is preferably scattered by cornea 120 and lens 132 and the scattered light imaged by a Scheimpflug camera 190. The Scheimpflug camera image may be analyzed, in accordance methods well known in the art, in order to derive at least one of the thickness of the cornea 120, the posterior radius of the cornea 120 and the anterior chamber depth. Here, by way of example, the corneal thickness of subject 116, the posterior radius of cornea 120 and the anterior chamber depth as measured by pachymeter 180 are shown to be respectively equal to 550 μm, 6.5 mm and 3.2 mm, displayed at a location 192 on display screen 172 of device 102.

Corneal topographer 182 preferably includes a placido disk 1100 preferably projecting a series of concentric rings onto a surface of cornea 120, as indicated by rays 1102. The reflection of these rings from the cornea 120 preferably propagates through second lens 164 and is preferably imaged by a camera 1104, and the resultant image analyzed in order to derive at least the anterior radius of the cornea 120. Here, by way of example, the anterior radius of cornea 120 as found by corneal topographer 182 is shown to be equal to 7.8 mm, displayed at a location 1106 on display screen 172 of device 102.

It is appreciated that not all of the above-described parameters measured by system 100 are necessarily directly measured thereby and that one or more of these parameters may alternatively be derived based on other measured parameters. For example, rather than the posterior radius of cornea 120 being measured by pachymeter 180, the posterior radius of cornea 120 may be estimated based on the measured value of the anterior radius of cornea 120 minus 1.25. Such an approach may be useful in the case that pachymeter 180 is embodied as an ultrasound pachymeter, which type of pachymeter is typically not capable of measuring the posterior radius of the cornea.

Furthermore, it is appreciated that the above-described configurations of refraction measurement module 112 and corneal measurement module 118 are exemplary only and that each of modules 112 and 118 may be modified by one skilled in the art to include additional or alternative components. Particularly, it is appreciated that the functionality of corneal measurement module 118, although illustrated and described herein as being distributed between pachymeter 180 and corneal topographer 182 may alternatively be carried out by a single corneal measurement system capable of providing measurements of at least both corneal shape and thickness.

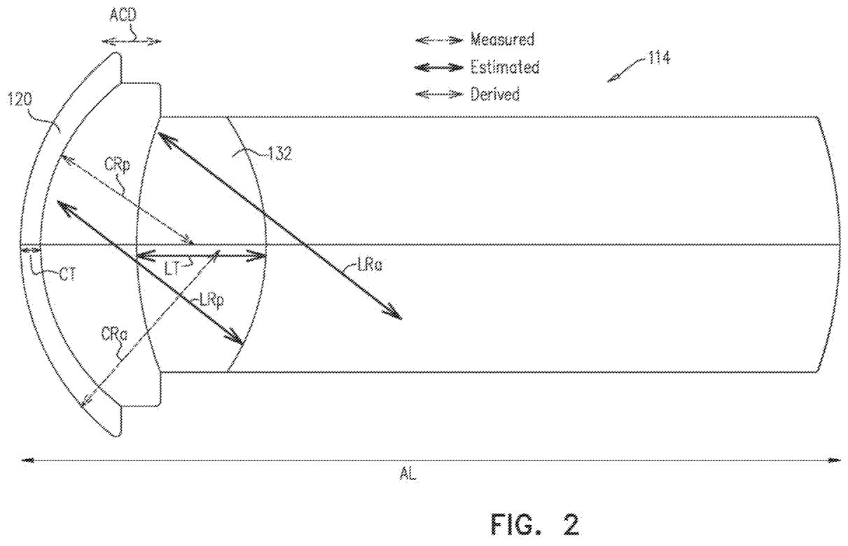

Parameters measured and output by measurement system 122 are indicated in FIG. 2, including the corneal thickness CT, the corneal anterior radius CRₐ and the corneal posterior radius CRₚ. In addition, pachymeter 180 may be used to measure the anterior chamber depth ACD, corresponding to the distance between the posterior corneal surface and the anterior surface of lens 132. The refraction of eye 114, as measured by refraction measurement subsystem 112, is not indicated in FIG. 2 due to the complexity of representing the refractive error of eye 114 by a single simple index.

It is appreciated that the configuration of measurement subsystem 122 as a single integrated measurement system, outputting the various measured parameters indicated in FIG. 2 by way of integrated measurement submodules, is a highly advantageous feature of the present invention. As a result of refraction measurement module 112 and corneal measurement module 118 being mutually integrated, the elements thereof are preferably internally calibrated together on a common calibration axis, thus reducing or eliminating centering errors that would otherwise typically arise should the measured parameters indicated in FIG. 2 be provided by separate, non-mutually integrated instruments. As appreciated from the foregoing description, second lens 164 is preferably common to all of the measurement paths of refraction measurement module 112 and corneal measurement module 118, thereby defining a common focus and working position for all of the measurement modules included in integrated measurement subsystem 122.

Furthermore, the use of a single integrated measurement subsystem 122 is highly convenient and leads to reduced errors in parameters measured thereby, in comparison to errors present in the same or similar parameters as respectively measured by separate, non-mutually integrated instruments. Additionally, since measurement subsystem 122 needs only be aligned once with subject 116, following which alignment all relevant parameters may be measured in one sitting, use of system 100 is highly time efficient, in comparison to the time that would be required to align a subject with a number of individual instruments, each measuring an individual parameter.

Measured parameters output by measurement subsystem 122 are preferably provided to axial length finding subsystem 130 to be used thereby for calculating the axial length of eye 114, the axial length being broadly defined as the distance between the anterior surface of cornea 120 and the fovea of the retina 144, which axial length AL is also indicated in FIG. 2.

In accordance with one preferred embodiment of the present invention, in addition to the measured parameters received from measurement subsystem 122, at least one modelled parameter of lens 132 of eye 114 is also taken into account by axial length finding system 130 in calculating the axial length of eye 114. Any appropriate ocular model may be used in order to estimate relevant parameters of lens 132.

Particularly preferably, parameters of lens 132 may be estimated based on an age-related lens model, in which case the age or birthdate of subject 116 is preferably supplied to system 100. An example of one possible age-related lens model suitable for use with preferred embodiments of the present invention is described in ‘Age related changes in optical and biometric characteristics of emmetropic eyes’, David A. Atchison et. al., Journal of Vision (2008) 8:4 (29) 1:20. Alternatively, parameters of lens 132 may be estimated based on a refractive error-related lens model, in which case the refraction as measured by refraction measurement module 112 is used in order to estimate parameters of lens 132. An example of one possible refractive error-related lens model suitable for use with preferred embodiments of the present invention is described in ‘On the prediction of optical aberrations by personalized eye models’, Rafael Navarro et. al., Optometry and Vision Science, 2006 83(6), 371-381. Axial length finding subsystem 130 preferably includes computerized systems for estimating at least one lens parameter, based on appropriate ocular models and relevant input data, including, by way of example, the age of subject 116 or the refractive error thereof. Such input data may be automatically supplied to axial length finding subsystem 130 by measurement subsystem 122 or may be input by a user operating device 102.

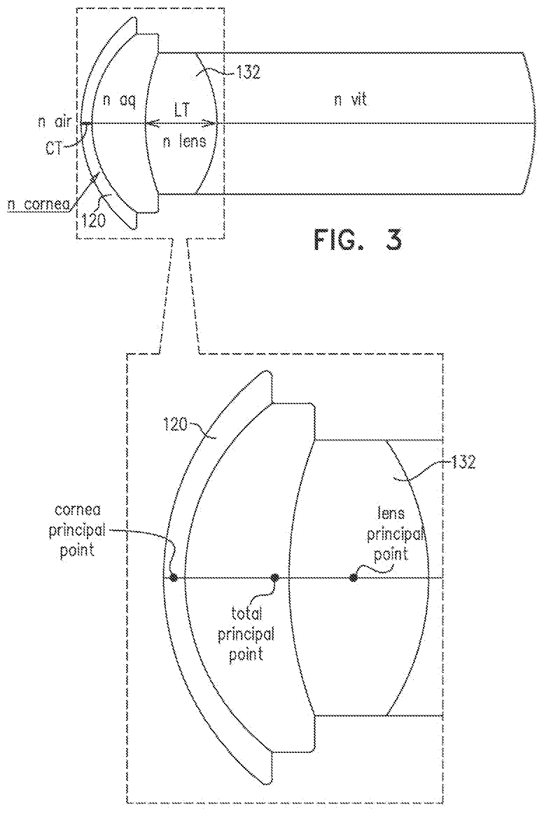

The above-described models, or any other suitable ocular models, may be used to generate at least one estimated parameter of lens 132. Particularly preferably, the lens anterior radius LRₐ lens posterior radius LR_{P} and lens thickness LT are estimated, as indicated in FIG. 2. Axial length finding subsystem 130 preferably calculates the axial length of eye 114 based on taking into account both the measured parameters provided by measurement subsystem 122 and the estimated parameters based on a model of lens 132. The axial length may be calculated using mathematical formulae, as is further detailed henceforth with reference to FIG. 3. The axial length may alternatively be calculated using ray tracing techniques, in accordance with methods known in the art. Here, by way of example, the axial length of eye 114 as output by axial length calculation module 130 is shown to be equal to 24 mm, displayed at a location 1120 on display screen 172 of device 102 in FIG. 1.

It is appreciated that the axial length of eye 114 thus may be calculated by the axial length finding subsystem of the present invention based on taking into account a combination of directly measured and estimated parameters of eye 114. Furthermore, the axial length is preferably found without requiring direct measurement thereof and rather is calculated based on other parameters, both measured and estimated, of eye 114.

Furthermore, it is understood that the accuracy of the axial length calculated by axial length calculation subsystem 130 is enhanced due to the taking into account of parameters of lens 132. It is appreciated that should parameters of lens 132 be ignored in the calculation of axial length performed by axial length calculation subsystem 130, the accuracy of the axial length found would be reduced.

Axial length as derived by system 100 may be useful for a variety of applications, particularly preferably including monitoring of myopia progression. It is appreciated that system 100 is particularly well suited for performance of repeated, follow up measurements due to the simplicity and cost-effectiveness thereof.

A possible mathematical formulaic technique that may be carried out by axial length calculation subsystem 130 for deriving the axial length of eye 114 based on measured parameters provided by measurement subsystem 122 and estimated lens parameters extracted from ocular models, may be best understood with reference to FIG. 3.

Turning now to FIG. 3, in which relevant parameters are indicated, at a first stage of the axial length calculation, the power of cornea 120 is preferably calculated. The power of the cornea P_{c} is preferably found in accordance with:

P_{c}=P_{1c}+P_{2c}−[CT×(P_{1c}×P_{2c})/n_{cornea}] (1)

where

P_{1c}=(n_{cornea}−nₐᵢᵣ)/CRₐ (2)

and

P_{2c}=(n_{aqueous}−n_{cornea})/CRₚ (3)

and where CT is the corneal thickness, as measured in micrometers by pachymeter 180, CRₐ and CRₚ are the corneal anterior and posterior radii respectively, as measured in millimeters by corneal topographer 182 and pachymeter 180 respectively, and n_{cornea}, nₐᵢᵣ and n_{aqueous} are respectively the refractive indices of the cornea, air and the aqueous humour, which values may be estimated using reference values well known in the art.

At a second stage of the axial length calculation, the power of lens 132 is preferably calculated. The power of the lens P_{L} is preferably found in accordance with:

P_{L}=P_{1L}+P_{2L}−[LT×(P_{1L}×P_{2L})/nₗₑₙₛ] (4)

where

P_{1L}=(nₗₑₙₛ−n_{aqueous})/LRₐ (5)

and

P_{2L}=(nᵥᵢₜᵣₑₒᵤₛ−nₗₑₙₛ)/LRₚ (6)

and where LT is the lens thickness, as estimated in millimeters based on a suitable ocular model, LRₐ and LRₚ are the lens anterior and posterior radii respectively, as estimated in millimeters based on a suitable ocular model, nᵥᵢₜᵣₑₒᵤₛ and n_{aqueous} are the refractive indices of the vitreous humour and the aqueous humour respectively, which values may be estimated using reference values well known in the art and nₗₑₙₛ is the refractive index of the lens, which may be estimated based on an age-related lens model.

At a third stage of the axial length calculation, the total combined power of the cornea 120 and lens 132 is preferably calculated. The total power Pₜₒₜₐₗ is preferably found in accordance with:

Pₜₒₜₐₗ=P_{c}+P_{L}−[d×(P_{c}×P_{L})/n_{aqueous}] (7)

where P_{c} and P_{L} and n_{aqueous} are as defined hereinabove and d is a distance between a cornea principal point PP_{cornea} and a lens principal point PPₗₑₙₛ, whereby d represents an effective length of the cornea 120—lens 132 system corrected for the principal points thereof.

In order to calculate d, which is unknown in the above equation, the total distance dₜₒₜₐₗ corresponding to the total length of the front part of eye 114 including CT, ACD and LT, may be found in accordance with:

dₜₒₜₐₗ=CT+ACD+LT (8)

where CT is the corneal thickness, as measured in micrometers by pachymeter 180, ACD is the anterior chamber depth, as measured in millimeters by pachymeter 180 and LT is the lens thickness, as estimated in millimeters based on a suitable ocular model.

In addition, the lens principal point PPₗₑₙₛ may be found by:

PPₗₑₙₛ=(nᵥᵢₜᵣₑₒᵤₛ/P_{L})×(LT/nₗₑₙₛ)×P_{1L} (9)

and the cornea principal point PP_{cornea} may be found by:

PP_{cornea}=(nₐᵢᵣ/P_{c})×(CT/n_{corea})×P_{2c} (10)

Utilizing equations (8)-(10), d may then be found in accordance with:

d=dₜₒₜₐₗ−PPₗₑₙₛ−PP_{cornea} (11)

where d is measured in millimeters.

It is understood that equations (1)-(11) serve to calculate the individual powers of various elements in eye 114 as well as the various distances therein.

At a fourth stage of the axial length calculation, all of the individual elements within eye 114 are preferably treated as a single optical element, acting at a total principal point PPₜₒₜₐₗ. The distance V from the principal point to the point of best focus, corresponding to the location of the retina, may then be calculated in accordance with:

V=nᵥᵢₜᵣₑₒᵤₛ/(Pₜₒₜₐₗ−(nₐᵢᵣ/u)) (12)

where u is the reciprocal of the sphere average defined as

sphere average=sphere+(cylinder/2) (13)

The distance V from the principal point to the point of best focus is then preferably corrected to give the distance V_{corrected} from the posterior surface of lens 132 to the retina, in accordance with:

V_{corrected}=V−PPₗₑₙₛ+PPₜₒₜₐₗ (14)

where

PPₜₒₜₐₗ=(nₐᵢᵣ/Pₜₒₜₐₗ)×(d/n_{aqueous})×P_{L} (15)

The axial length AL may then be found in accordance with:

AL=V_{corrected}+LT+ACD+CT (16)

It is understood that the highly simplified formulaic approach described hereinabove is based on modelling the optical system formed by the eye in accordance with Gaussian optics. It is appreciated that such a technique advantageously allows the calculation of axial length using simple, formulaic calculations which may be rapidly automatically carried out by axial length finding subsystem 130 by computerized functions thereof and preferably involve little computing power.

Furthermore, it is appreciated that the various calculation stages need not necessarily be performed in the order listed and may be interchanged or interfaced by additional steps, for further refining or correcting calculated or estimated values.

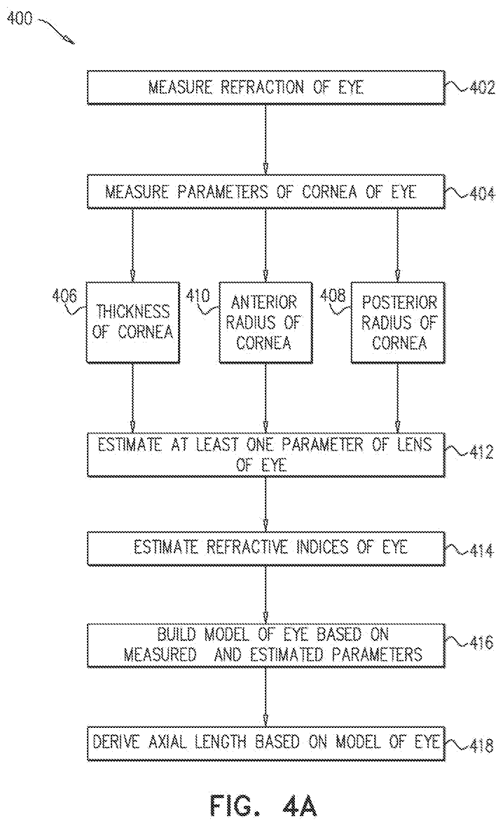

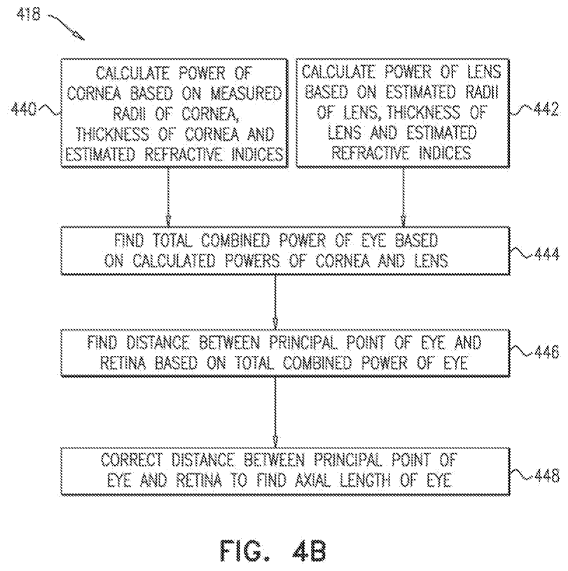

Reference is now made to FIGS. 4A and 4B, which are simplified flow charts respectively illustrating steps in the derivation of the axial length of the eye of a subject, based on taking into account parameters indicated in FIG. 2;

As seen in FIG. 4A, an ocular measurement process 400 may begin at a first refraction measurement step 402, as which step 402 the refraction of the eye of a subject is measured. By way of example, the refraction of the eye may be measured by a Shack-Hartmann refraction measurement system such as module 112 illustrated in FIG. 1, or by any other suitable refraction measurement device.

At a second step 404, various parameters of the cornea of the eye of the subject are additionally measured. Such parameters may include the thickness of the cornea and the corneal posterior radius, as may be measured by a pachymeter such as pachymeter 180 and respectively indicated at steps 406 and 408, and the corneal anterior radius, as may be measured by a corneal topographer such as corneal topographer 182, as indicated at a step 410. It is appreciated that step 404 is not limited to the measurement of those parameters indicated at steps 406, 408 and 410 and may include the measurement of additional or alternative parameters of the cornea, including the anterior chamber depth, by way of example.

At a third step 412, various parameters of the lens of the eye of the subject are preferably estimated. Estimated lens parameters may include the lens anterior and posterior radii, lens thickness and lens refractive index, in addition to other possible parameters of interest. Lens parameter estimation step 412 may include estimation of parameters of the lens based on any suitable ocular model, including age-based lens models and refraction based lens models, as are well known in the art.

At a fourth step 414 various refractive indices of the eye are preferably estimated, preferably including refractive indices of the cornea, aqueous humour and vitreous humour. These refractive indices may be based on standard reference values of these parameters, as are well known in the art.

It is appreciated that although steps 402, 404, 412 and 414 are illustrated and described as being performed respectively sequentially, steps 402, 404, 412 and 414 may be at least partially reordered or may be performed at least partially simultaneously with respect to one another.

At a fifth step 416, a model of the eye is preferably constructed based on the measured and estimated parameters found at steps 402-414 and the axial length of the eye may be found based on the model at a sixth step 418. It is appreciated that the construction of a model of the eye and derivation of the axial length based thereon may be carried out by an axial length calculation subsystem, such as axial length finding system 130 of FIG. 1.

Turning now to FIG. 4B, a preferred method for carrying out sixth axial length calculation step 418 is shown. As seen at a first axial length calculation sub-step 440, the power of the cornea may be calculated based on the anterior and posterior radii of the cornea, as measured at step 404, and relevant estimated refractive indices, as found at step 414. By way of example, first axial length calculation sub-step 440 may involve the performance of calculations set forth in equations (1)-(3) hereinabove.

Following, concurrent with or preceding first sub-step 440, the power of the lens may be calculated at a second sub-step 442. The power of the lens may be calculated based on the estimated parameters of the lens, as found at step 412, and relevant estimated refractive indices, as found at step 414. By way of example, second sub-step 442 may involve the performance of those calculations set forth in equations (4)-(6) hereinabove.

The total combined power of the eye may then be calculated, based on the calculated powers of the lens and cornea, as seen at a third sub-step 444. By way of example, third sub-step 444 may involve the performance of those calculations set forth in equations (7)-(11) hereinabove.

As seen at a fourth sub-step 446, the distance from the principal point of the eye to the point of best focus, coincident with the retina, is then preferably found. The principal point of the eye corresponds to that theoretical point at which the optical system formed by all of the individual optical components of the eye may be considered to effectively act. By way of example, fourth sub-step 446 may involve the performance of the calculation set forth in equations (12)-(13) hereinabove.

Following the finding of the distance between the principal point of the eye and the retina, this distance is then preferably corrected to yield the distance between the posterior lens surface to the retina and hence the distance between the cornea and the retina, corresponding to the axial length of the eye, as seen at a fifth sub-step 448. By way of example, fifth sub-step 448 may involve the performance of the calculations set forth in equations (14)-(16) hereinabove.

It is appreciated that the calculations described with reference to steps 440-448 hereinabove may be performed in an automated, computerized manner, for example by axial length finding subsystem 130 of FIG. 1. It is further appreciated that the sixth axial length calculation step 418 may alternatively involve steps other than steps 440-448, including, but not limited to, ray tracing techniques.

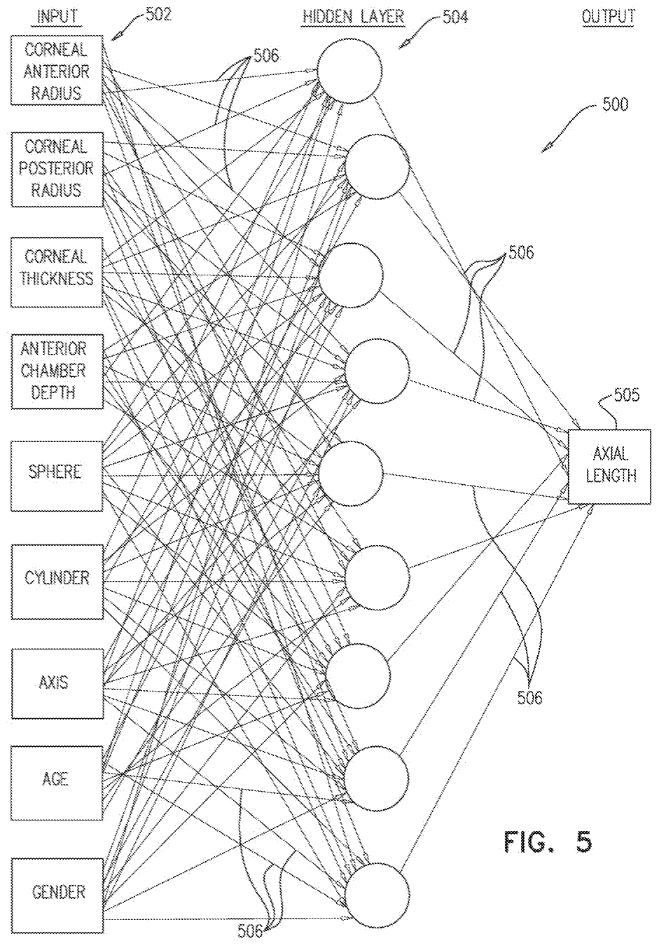

Reference is now made to FIG. 5, which is a simplified schematic illustration of a neural network forming part of a system of the type shown in FIG. 1, constructed and operative in accordance with another preferred embodiment of the present invention.

As seen in FIG. 5, there is provided a neural network 500, preferably incorporated in axial length finding system 130 (FIG. 1). Neural network 500 may be employed to find the axial length of eye 114 (FIG. 1) by way of machine learning, based on various input parameters as measured and provided thereto by measurement subsystem 122 (FIG. 1). It is understood that the finding of the axial length of eye 114 using machine learning techniques, such as those performed by neural network 500, represents an alternative embodiment of axial length finding subsystem 130 to that described hereinabove with reference to FIGS. 3-4B.

Neural network 500 may operate in an initial training mode or an active output mode. In an initial training mode, a variety of measured parameters 502, preferably measured by measurement subsystem 122 are preferably provided as input parameters to neural network 500. Measured parameters 502 may include the corneal anterior radius, corneal posterior radius, corneal thickness, anterior chamber depth and the refractive error of eye 114 including sphere, cylinder and axis. It is understood that such parameters may be conveniently provided to neural network 500 included in axial length finding subsystem 130 by integrated measurement subsystem 122 or may be measured by other alternative devices. Additionally, measured parameters 502 may include the age and gender of subject 116 (FIG. 1), which data may be input by a user or may be automatically supplied. Additional parameters may also be included in measured parameters 502, such as higher order Zernike ocular and corneal coefficients and the irido angle.

In a training mode, for each set of parameters 502 the corresponding measured axial length is also preferably supplied. The axial length corresponding to the input measured parameters 502 may be measured by any suitable methods known in the art, including by way of an ultrasound or Optical Coherence Tomography biometer.

An algorithm 504 is then preferably generated to relate the sets of measured parameters 502 to the corresponding measured axial length, by way of a learning process. The algorithm 504 is represented in FIG. 5 as a hidden layer, receiving inputs in the form of measured parameters 502 from an input layer and relating the received inputs to the measured axial length at an output layer 505. The inclusion of a greater number of input measured parameters 502 within each set of input measurements may, but does not necessarily, increase the accuracy of the learning process carried out by neural network 500.

A multiplicity of arrows 506 extending between the input layer and hidden layer and between the hidden layer and output 505 symbolically represent neurons or mathematical functions forming a part of algorithm 504. Each mathematical function as represented by a single one of arrows 506 preferably has an associated threshold, weighting value and activation value, determining under what conditions the given function is activated and how. The threshold, weighting and activation values of each function, as well as the function itself, are preferably found and set as part of the learning process carried out by algorithm 504. It is understood that the mathematical functions represented by arrows 506 are depicted in a highly simplified symbolic manner in FIG. 5 in order to represent the general arrangement thereof.

The greater the number of sets of input measurement parameters 502 and corresponding outputs supplied to algorithm 504, the greater the accuracy of the learning process and the hence the more accurate the derived relationship between the input measured parameters and the output axial length. The actual number of measurements required to be supplied to neural network 500 in order to complete the training thereof depends on the number of input measured parameters 502 as well as the required accuracy of the axial length output. By way of example, neural network 500 may require over 1000 sets of input measurements in order to be sufficiently accurately trained.

Algorithm 504 may involve a single layer of a learning process between the input and output stages, as illustrated in FIG. 5. Alternatively, algorithm 504 may involve deep learning in which multiple layers of a learning process are present between the input and output stages, which multiple layers may, but do not necessarily, increase the accuracy of the learning process.

Following the completion of training of neural network 500, neural network 500 may then operate in an active output mode. In the active output mode of neural network 500, the mathematical functions learned in the training mode of neural network 500 are applied to newly measured input parameters 502 in order to derive an output corresponding axial length via the hidden layers. It is appreciated that in an active output mode, neural network 500 is preferably static, in that the mathematical functions 506 forming part of algorithm 504 are no longer being refined by learning, but rather have been set and are simply being applied to input measured parameters.

It is appreciated that the use of machine learning techniques for deriving axial length, such as those carried out by neural network 500, does not require the inclusion of any estimated parameters in order to derive the axial length, but rather finds a link between the measured input parameters and the output axial length. It is further appreciated that machine learning techniques for the finding of the axial length, as carried out by axial length finding system 130, are not limited to neural networks and may involve other machine learning, including deep learning, techniques.

It will also be appreciated by persons skilled in the art that the present invention is not limited by what has been particularly claimed hereinbelow. Rather, the scope of the invention includes various combinations and subcombinations of the features described hereinabove as well as modifications and variations thereof as would occur to persons skilled in the art upon reading the forgoing description with reference to the drawings and which are not in the prior art.

1. An ocular measurement system comprising:
an integrated measurement subsystem comprising at least a refraction measurement module for measuring at least refraction of an eye of a subject and a corneal measurement module integrated with said refraction measurement module for measuring at least a thickness and a shape of a cornea of said eye; and
an axial length finding subsystem for finding an axial length of said eye, based on taking into account at least said refraction of said eye and said thickness and said shape of said cornea.

2. An ocular measurement system according to claim 1, wherein said finding said axial length of said eye additionally comprises taking into account a modelled value of at least one parameter of a lens of said eye.

3. An ocular measurement system according to claim 2, wherein said at least one parameter of said lens comprises at least one of a thickness of said lens, a radius of an anterior surface of said lens, a radius of a posterior surface of said lens and a refractive index of said lens.

4. An ocular measurement system according to claim 1, wherein said refraction measurement module comprises a Shack-Hartmann system for analyzing a wavefront of light reflected from said eye.

5. An ocular measurement system according to claim 1, wherein said corneal measurement module comprises at least a pachymeter and a corneal topographer.

6. An ocular measurement system according to claim 5, wherein said pachymeter measures at least one of a thickness of said cornea, a posterior radius of said cornea and an anterior chamber depth of said eye.

7. An ocular measurement system according to claim 5, wherein said corneal topographer measures at least an anterior radius of said cornea.

8. An ocular measurement system comprising:
a refraction measurement subsystem for measuring refraction of an eye of a subject;
a corneal measurement subsystem for measuring at least a thickness and a shape of a cornea of said eye; and
an axial length calculation subsystem for performing a formulaic calculation for calculating an axial length of said eye, based on taking into account at least said refraction of said eye, said thickness and said shape of said cornea and a modelled value of at least one parameter of a lens of said eye.

9. An ocular measurement system according to claim 8, wherein said at least one parameter of said lens comprises at least one of a thickness of said lens, a radius of an anterior surface of said lens, a radius of a posterior surface of said lens and a refractive index of said lens.

10. An ocular measurement system according to claim 8, wherein said formulaic calculation comprises at least one formulaic calculation for finding at least one of a power of said cornea, a power of said lens, a combined power of said cornea and said lens and a distance between a principal point of said eye and a focal point of said eye.

11. An ocular measurement system according to claim 10, wherein said power of said cornea is calculated in accordance with P_{c}=P_{1c}+P_{2c}−[CT×(P_{1c}×P−_{2c})/n_{cornea}], where P_{c} is said power of said cornea, P_{1c}=(n_{cornea}−nₕₐᵢᵣ)/CRₐ, P_{2c}=(n_{aqueous}−n_{cornea})/CRₚ, CT is a thickness of said cornea, n_{cornea} is a refractive index of said cornea, nₐᵢᵣ is a refractive index of air, CRₐ is an anterior radius of said cornea, n_{aqueous} is a refractive index of an aqueous humour of said eye and CRₚ is a posterior radius of said cornea.

12. An ocular measurement system according to claim 11, wherein said power of said lens is calculated in accordance with P_{L}=P_{1L}+P_{2L}−(LT×(P_{1L}×P_{2L})/nₗₑₙₛ] where P_{L} is said power of said lens, P_{1L}=(nₗₑₙₛ−n_{aqueous})/LRₐ, P_{2L}=(nᵥᵢₜᵣₑₒᵤₛ−nₗₑₙₛ)/LRₚ, LT is a thickness of said lens, LRₐ is an anterior radius of said lens, LRₚ is a posterior radius of lens, nₗₑₙₛ is a refractive index of said lens and nᵥᵢₜᵣₑₒᵤₛ is a refractive index of a vitreous humour of said eye.

13. An ocular measurement system according to claim 12, wherein said combined power of said cornea and said lens is calculated in accordance with Pₜₒₜₐₗ=P_{c}+P_{L}−[d×(P_{c}×P_{L})/n_{aqueous}], where Pₜₒₜₐₗ is said combined power of said cornea and said lens, d is an effective length of a cornea lens system and is calculated in accordance with d=dₜₒₜₐₗ−PPₗₑₙₛ−PP_{cornea}, wherein dₜₒₜₐₗ=CT+ACD+LT, wherein ACD is an anterior chamber depth, PPₗₑₙₛ is a principal point of said lens, defined as PPₗₑₙₛ=(nᵥᵢₜᵣₑₒᵤₛ/P_{L})×(LT/nₗₑₙₛ)×P_{1L} and PP_{cornea} is a principal point of said cornea, defined as PP_{cornea}=(nₐᵢᵣ/P_{c})×(CT/n_{cornea})×P_{2c}.

14. An ocular measurement system according to claim 13, wherein said distance between a principal point of said eye and a focal point of said eye is calculated in accordance with V=nᵥᵢₜᵣₑₒᵤₛ/(Pₜₒₜₐₗ−(nₐᵢᵣ/u), where V is said distance between a principal point of said eye and a focal point of said eye and u is a reciprocal of a sphere average of said eye.

15. An ocular measurement system comprising:
a refraction measurement subsystem for measuring refraction of an eye of a subject and providing a measured refraction output;
a corneal measurement subsystem for measuring at least a thickness and a shape of a cornea of said eye and providing a measured corneal thickness and shape output; and
an axial length finding subsystem receiving at least said measured refraction output and said measured corneal thickness and shape output and employing machine learning for finding an axial length of said eye.

16. An ocular measurement system according to claim 15, wherein said refraction measurement subsystem is integrated with said corneal measurement subsystem.

17. An ocular measurement system according to claim 15, wherein said machine learning comprises employing an artificial neural network.

18. An ocular measurement system according to claim 15, wherein said machine learning comprises deep learning.

19. An ocular measurement system according to claim 15, wherein said refraction measurement subsystem comprises a Shack-Hartmann system for analyzing a wavefront of light reflected from said eye and said corneal measurement subsystem comprises at least a pachymeter and a corneal topographer.

20. An ocular measurement system according to claim 15, wherein said axial length finding subsystem is additionally provided with other input parameters relating to said subject, said other input parameters comprising at least one of an age of said subject, a gender of said subject and an anterior chamber depth of said eye of said subject.
